(19) United States
(12) Patent Application Publication
(10) Pub. No.: US 2011/0223246 A1
(43) Pub. Date: Sep. 15, 2011

(54) DOCOSAHEXAENOIC ACID BOUND IN PHOSPHOLIPIDS AND METHOD OF RECOVERING SAME FROM A NATURAL SOURCE

(76) Inventor: Joar Opheim, Aptos, CA (US)

(21) Appl. No.: 13/038,216

(22) Filed: Mar. 1, 2011

Related U.S. Application Data
(60) Provisional application No. 61/312,527, filed on Mar. 10, 2010.

(51) Int. Cl. A61K 36/53; A61K 31/685; A61K 31/683; A61K 9/48; C11B 1/06; A61P 25/00; A61P 27/02

(52) U.S. Cl. 424/456; 514/77; 514/121; 554/8; 424/745

(57) ABSTRACT

Medicaments and therapeutic compositions contain (1) phospholipids having 4,7,10,13,16,19-docosahexaenoic acid covalently bound thereto and (2) at least one omega-3 polyunsaturated fatty acid, or at least one pharmaceutically acceptable omega-3 polyunsaturated fatty acid derivative or mixtures thereof, wherein about 25% of the total 4,7,10,13,16,19-docosahexaenoic acid moieties in the composition are covalently bound to phospholipids, and wherein components (1) and (2) are present in amounts effective to support overall neurological health in a subject.

### CROSS-REFERENCES TO RELATED APPLICATION

This application claims priority to U.S. Provisional No. 61/312,527, filed Mar. 10, 2010, commonly owned, incorporated by reference herein for all purposes.

### BACKGROUND OF THE INVENTION

The present invention relates to compositions employed in therapeutic compositions, nutritional supplements and/or medicaments wherein the compositions comprise DHA polyunsaturated omega-3 fatty acid covalently bound in phospholipids (referred to herein as “DHA phospholipids” or “PL-form DHA”) and a method of recovering such DHA phospholipids from a natural source. The DHA phospholipids can be used in compositions which can be administered to a subject to promote, support or maintain neurological, retinal or reproductive health.

### DESCRIPTION OF THE PRIOR ART

WO 2008/142482 A2, published on Nov. 27, 2008 with Pronova Biopharma Norge AS as the applicant, discloses compositions containing omega-3 lipid compounds substituted at their 2-positions. Some of these substituted compounds can be phospholipids. The compounds are said to have therapeutic activity in a number of areas.

WO 2008/060163 A1, published May 22, 2008 with Pronova Biopharma Norge AS as the applicant, discloses a process for production of omega-3 rich marine phospholipids from krill. The process involves extracting a substantially total lipid fraction from fresh krill by reducing the water content of krill raw material, and isolating the lipid fraction. The process can include washing the krill with ethanol, methanol, propanol or isopropanol and isolating the lipid fraction from the alcohol. The krill can be heated to 60-100° C. before washing.

U.S. Pat. No. 5,336,792, issued Aug. 9, 1994 to Sola et al., discloses a process to enrich fat with polyunsaturated fatty acids and phospholipids by forming a presscake of iced fjord herring containing polyunsaturated fatty acids and phospholipids. The presscake is dissolved in a fat dissolving polar alcohol, the solids are separated, and the liquid evaporated until a precipitation of a first fat fraction occurs. The first fat fraction is separated and evaporation resumed until the precipitation of a second fat fraction. The second fat fraction, with a higher content of polyunsaturated fatty acids and phospholipids than the first fat fraction, is separated from the remainder of the solution.

U.S. Pat. No. 7,189,418, issued Mar. 13, 2007 to Hiratsuka et al., discloses a method for extracting a lipid mixture having a high percentage of phospholipids comprising polyunsaturated fatty acids. The method comprises the steps of (a) heating the viscera of fish with hot water or steam, and (b) extracting from the heated viscera, using a solvent, the lipid mixture containing phospholipids comprising polyunsaturated fatty acids. The viscera may be dried after processing in hot water of 60° C. or higher, and after the drying, extraction with a solvent may be performed. The lipid mixture contains phosphatidylserine comprising docosahexaenoic acid in high concentration.

U.S. Patent Application Publication No. 2008/0085320 A1, published Apr. 10, 2008 by Dror, discloses glycerolphospholipids having long chain polyunsaturated fatty acids covalently bound to them. The glycerolphospholipids are said to be useful in the treatment of various cognitive and mental conditions and disorders and for maintenance of normal functions of brain-related systems and processes.

### BRIEF SUMMARY OF THE INVENTION

Provided in accordance with the present invention is a therapeutic composition comprising (1) phospholipids having 4,7,10,13,16,19-docosahexaenoic acid covalently bound thereto and (2) at least one omega-3 polyunsaturated fatty acid, or at least one pharmaceutically acceptable omega-3 polyunsaturated fatty acid derivative or mixtures thereof, wherein about 25% of the total 4,7,10,13,16,19-docosahexaenoic acid moieties in the composition are covalently bound to phospholipids, and wherein components (1) and (2) are present in amounts effective to support overall neurological health in a subject.

In some embodiments, the therapeutic compositions may be compositions wherein component (1) further comprises phospholipids having 5,8,11,14,17-eicosapentaenoic acid covalently bound thereto. In some embodiments about 29% of the total 5,8,11,14,17-eicosapentaenoic acid moieties in the composition are covalently bound to phospholipids.

In some embodiments, the therapeutic compositions may be compositions wherein the omega-3 polyunsaturated fatty acid derivatives are glycerides. In some embodiments, the omega-3 polyunsaturated fatty acid derivatives are derivatives of EPA, derivatives of DHA or mixtures of derivatives of EPA and derivatives of DHA.

Further provided in accordance with the present invention are compositions wherein component (2) is a mixture comprising about 35 wt. % triglycerides of EPA and about 25 wt. % triglycerides of DHA.

The present invention further provides a therapeutic composition wherein component (2) is a mixture comprising at least about 60 wt. % of a combination of EPA and DHA in a weight ratio of EPA:DHA of from about 1.4:1 to about 1.1:7, wherein the combination is at least about 60% in the triglyceride form of the EPA and DHA and the balance is at least about 80% mono- and di-glycerides. Also provided are compositions wherein the combination comprises about 10 wt. % triglycerides of EPA and about 50 wt. % triglycerides of DHA or wherein the combination comprises about 11 wt. % triglycerides of EPA and about 70 wt. % triglycerides of DHA. Also provided are compositions wherein the combination is at least about 80% in the triglyceride form, at least about 90% in the triglyceride form, at least about 98% in the triglyceride form, or least about 98% in the triglyceride form and the remainder is monoglycerides, diglycerides or both. The present invention further provides therapeutic compositions wherein the combination comprises about 15 wt. % triglycerides of EPA and about 40 wt. % triglycerides of DHA.

The present invention also provides a dose of the medicament or therapeutic composition wherein the dose of medicament or therapeutic composition comprises about 200 mg to about 6 grams of derivatives of DHA or derivatives of DHA plus derivatives of EPA wherein about 5% to about 99% of the derivatives are phospholipids and the remainder are derivatives other than phospholipids such as glycerides (i.e., monoglycerides, diglycerides, triglycerides or mixtures thereof) or alkyl esters (e.g., methyl or ethyl esters).

Further provided in accordance with the present invention is a method of supporting overall neurological health in a subject comprising administering to the subject a dosage comprising the therapeutic composition of the present invention.

Also provided by the present invention is a method of supporting overall retinal health in a subject comprising administering to the subject a dosage comprising the therapeutic composition of the present invention.

Further provided by the present invention is a method of supporting overall reproductive health and/or fertility in a subject comprising administering to the subject a dosage comprising the therapeutic composition of the present invention.

Further provided by the present invention is a method of collecting phospholipids having 4,7,10,13,16,19-docosahexaenoic acid covalently bound thereto from herring roe, the method comprising:
(a) boiling a mixture comprising herring roe and water at about 100° C.,
(b) pressing the product of step (a) until excessive water is removed from the product and a presscake having a water content of 10% or less is formed,
(c) treating the presscake of step (c) with an alcohol until the phospholipids are released from it,
(d) collecting the released phospholipid molecules and distilling off the alcohol from the phospholipids to produce an oily product containing the phospholipid molecules, and
(e) deodorizing the product of step (d).

### DETAILED DESCRIPTION OF THE INVENTION

The compositions of the present invention provide the omega-3 fatty acid docosahexaenoic acid (“DHA”) in phospholipid (“PL”) form. DHA is a polyunsaturated fatty acid having a 22 carbon chain having six cis double bonds in it, with the first double bond from the omega end at the third carbon from the omega end. The chemical name for DHA is 4,7,10,13,16,19-docosahexaenoic acid (22:6 (n-3). They can also provide the omega-3 fatty acid eicosapentaenoic acid (“EPA”) in PL form. EPA is a polyunsaturated fatty acid having a 20 carbon chain having five cis double bonds in it, with the first double bond from the omega end at the third carbon from the omega end. The chemical name for EPA is 5,8,11,14,17-eicosapentaenoic acid ((20:5 (n-3). This is in contrast to other compositions derived from fish oil that provide DHA and EPA in the triglyceride (“TG”) form. Under healthy conditions, the body is able to turn TG-form DHA and TG-form EPA into the PL-form during digestion. However, in some individuals with poor digestion and/or some neurological conditions, this process is impaired and PL-form DHA and PL-form EPA are not adequately produced or retained. The compositions of this invention provide a good source of preformed PL-form DHA and PL-form EPA for such individuals in which PL-form DHA and EPA are not formed, retained and/or absorbed sufficiently.

In contrast to other PL-form omega-3 polyunsaturated fatty acid-containing dietary supplements that are derived from krill, the compositions of the present invention are derived from wild herring roe oil. This provides two main advantages. First, the wild herring roe oil yields more omega-3 polyunsaturated fatty acids per serving. Whereas krill based products yield less than 75 mg PL-form EPA and 45 mg PL-form DHA per recommended serving, the compositions of this invention provide more than 120 mg of PL-form EPA and 450 mg PL-form DHA per recommended serving. Second, whereas krill-based PL-form products provide mainly PL-form EPA and much less PL-form DHA, wild herring roe oil yields much more PL-form DHA. This is a critical difference, given that PL-form DHA is the main fatty acid found in neurological structures, such as the brain, and in the retina and sperm. Given the high concentration of PL-form DHA in brain and nerve, retinal, and reproductive cells, the compositions of the present invention may support increased absorption, delivery and retention of DHA in these key areas of the body.

In some embodiments, the therapeutic compositions of this invention include compositions derived from fish oil in which the fish oil comprises at least about 60% of omega-3 oils, or at least about 70% omega-3 oils. (As used herein, the term “about” means that the value to which it refers can vary slightly, such as by 5% or 10%.) In some embodiments, the therapeutic compositions include compositions in which the omega-3 oils comprise about 10% EPA derivative and about 50% DHA derivative, or in which the omega-3 oils comprise about 11% EPA derivative and about 70% DHA derivative. In some embodiments, the therapeutic compositions comprise a daily dose of the therapeutic compositions which is delivered by an integral number of capsules.

In some embodiments, the daily dose of therapeutic composition comprises about 200 mg to about 6 grams of derivatives of DHA or derivatives of DHA plus derivatives of EPA wherein about 5% to about 99% of the derivatives are phospholipids and the remainder are derivatives other than phospholipids such as glycerides (i.e., monoglycerides, diglycerides, triglycerides or mixtures thereof) or alkyl esters (e.g., methyl or ethyl esters). In some embodiments, the therapeutic composition further comprises an antioxidant. In some embodiments, the antioxidant is chosen from the group consisting of rosemary, vitamin E, astaxanthine, carnitine, and ascorbyl palmitate.

### Phospholipids

Phospholipids are a class of lipids and are a major component of all biological membranes. All phospholipids contain a diglyceride, a phosphate group, and a simple organic molecule such as choline. In the DHA PLs of the present invention the DHA moiety is attached to the diglyceride portion of the phospholipid at the sn-2 site.

The DHA PLs of this invention can be phosphatidyl cholines, phosphatidyl serines, phosphatidyl ethanolamines, phosphatidyl inositols, lyso-phosphatidyl cholines or sphingomylins.

### Omega-3 Polyunsaturated Fatty Acids

As used herein, the term “omega-3 polyunsaturated fatty acid(s)” refers to a family of unsaturated fatty carboxylic acids that have in common a carbon-carbon bond in the n-3 position (i.e., the third bond from the methyl end of the molecule). Typically, they contain from about 16 to about 24 carbon atoms and from three to six carbon-carbon double bonds. Omega-3 polyunsaturated fatty acids can be found in nature, and these natural omega-3 polyunsaturated fatty acids frequently have all of their carbon-carbon double bonds in the cis-configuration.

Examples of omega-3 polyunsaturated fatty acids include, but are not limited to, 7,10,13-hexadecatrienoic acid (sometimes abbreviated as 16:3 (n-3)); 9,12,15-octadecatetrienoic acid (α-linolenic acid (ALA), 18:3 (n-3)); 6,9,12,15-octadecatetraenoic acid (stearidonic acid (STD), 18:4 (n-3)); 11,14,17-eicosatrienoic acid (eicosatrienoic acid (ETE), 20:3 (n-3)); 8,11,14,17-eicosatetraenoic acid (eicosatetraenoic acid (ETA), 20:4 (n-3)); 5,8,11,14,17-eicosapentaenoic acid (eicosapentaenoic acid (EPA), (20:5 (n-3)); 7,10,13,16,19-docosapentaenoic acid (docosapentaenoic acid (DPA), 22:5 (n-3)); 4,7,10,13,16,19-docosahexaenoic acid (docosahexaenoic acid (DHA), 22:6 (n-3)); 9,12,15,18,21-tetracosapentaenoic acid (tetracosapentaenoic acid, 24:5 (n-3)); and 6,9,12,15,18,21-tetracosahexaenoic acid (tetracosahexaenoic acid, 24:6 (n-3)).

Eicosapentaenoic acid (EPA) and docosahexaenoic acid (DHA) are found in nature in fish oils and other natural sources, and have been used in a variety of dietary/therapeutic compositions. EPA and DHA and/or their derivatives are preferred omega-3 polyunsaturated fatty acids and derivatives in the present invention.

The terms “EPA” and “DHA” are used herein in two contexts. When used in the context of omega-3 polyunsaturated fatty acid derivatives, “EPA” and “DHA” refer to the fact that the derivative contains an eicosapentaenoic acid moiety or docosahexaenoic acid moiety which is present as, for example, an ester or glyceride (e.g., mono-, di- and/or tri-glycerides). When used in the context of phospholipids, “EPA” and “DHA” refer to the fact that the EPA or DHA is covalently bonded to a phospholipid.

### Omega-3 Polyunsaturated Fatty Acid Derivatives

As used herein, the term “omega-3 polyunsaturated fatty acid derivative(s)” refers to omega-3 polyunsaturated fatty acids that have been reacted with another compound or otherwise modified so that the omega-3 polyunsaturated fatty acid no longer contains a free carboxylic acid. Examples of omega-3 polyunsaturated fatty acid derivatives include salts, esters (such as alkyl esters including, but not limited to, methyl and ethyl esters) and glycerides of omega-3 polyunsaturated fatty acids.

The omega-3 polyunsaturated fatty acid derivatives should be in a pharmaceutically acceptable form. As used herein, the term “pharmaceutically acceptable” means that the material to which it refers is not harmful to the subject.

As used herein, the term “glyceride” means a glycerol molecule (i.e., OHCH₂CHOHCH₂OH) in which one, two or all three of the hydroxyls have been esterified with a carboxylic acid, e.g., an omega-3 polyunsaturated fatty acid. Thus, “triglyceride” refers to glycerides in which all three hydroxyls on the glycerol have been esterified with (the same or different) carboxylic acids. “Diglyceride” refers to glycerides in which only two of the hydroxyls on the glycerol have been esterified with (the same or different) carboxylic acids. “Monoglyceride” refers to glycerides in which only one hydroxyl on the glycerol has been esterified with a carboxylic acid.

Omega-3 fatty acids can be found in nature in the triglyceride form (a glycerol with three fatty acids attached). The natural triglyceride form as found in raw fish oil cannot be readily separated as it occurs into purified EPA/DHA-containing mixtures by ordinary means such as distillation or crystallization, because the fatty acids are non-uniformly distributed among the triglyceride molecules. There are very few, if any, single triglyceride molecules which are composed of either three EPA moieties or three DHA moieties. Typically, there is a DHA moiety, an EPA moiety, and another fatty acid moiety in a triglyceride molecule. So in order to purify fatty acids to increase the proportion of EPA, DHA, or the total fraction of omega-3's, it is necessary to hydrolyze the triglycerides to remove at least some fatty acids from the glycerol.

The triglycerides may be converted by any method known to one skilled in the art without limitation. For example, the triglycerides may be converted by lipase-catalyzed esterification or lipase catalyzed acidolysis with ethyl or lauryl alcohol, which can selectively leave the highest amount of EPA and DHA bonded to glycerols and remove other components, leaving EPA and/or DHA as mono- or di-glycerides. The mono- and di-glycerides can then be separated into fractions with different EPA/DHA ratios, by methods familiar to those skilled in the art such as multiple stage vacuum distillation and/or fractional crystallization in urea. Advantageously, the purified EPA and DHA esters, after concentration, can be reattached to glycerol molecules using enzymatic reacylation to recreate glycerides which are otherwise identical to the original natural triglycerides, except that they are more concentrated in EPA and DHA combined, and they may also have a different ratio of EPA:DHA than the original fish oil. In some embodiments, at least 60% of the omega-3 fatty acids, and preferably 70% or more are converted to the triglyceride form in the reacylation process. The process may be successively repeated with addition of additional catalyst and/or enzyme and additional EPA and DHA until the desired specification proportions are met. About 60% of triglycerides can be made in the first pass of reacylation, with most of the remainder of the product being mono- and di-glycerides.

Polyunsaturated fatty acid triglycerides can be prepared using the following method.

1. Removal of Free Fatty Acids

Raw fish oil in the natural triglyceride molecular form which contain about 18% EPA and 12% DHA is heated to 60° C. to decrease viscosity. Sodium oxide is added to bind with free fatty acids in the oil. The mixture is moved to a separator where sodium oxide bound to free fatty acids (soap) floats to the top and is removed.

The oil is then moved to a second separator where warm water is preferably added to help remove traces of sodium oxide, as sodium oxide partitions to water, yet does not interact with the fish oil.

Citric acid may then be added to support splitting the oil from the combination of water and sodium oxide. The oil is then cooled to 30° C. to protect it from oxidation.

2. Stripping and Purification

Oil is moved to a separate stripping tank, and heated to 200° C. Ethyl esters can be added to support the removal of impurities, which bind to ethyl esters. Impurities such as dioxins, heavy metals, PCBs, fire retardants, furans and others evaporate and are drawn to the middle of the tank where a refrigerating element cools them down and drain them. The added esters are also removed with the impurities.

3. Esterification

The oil is moved to an esterification tank. Ethanol and sodium metal are added. Sodium metal is a catalyst for breaking off fatty acid strands from the glycerol backbone of the triglyceride fatty acid molecule, the free fatty acids then combined with ethanol to form ethyl esters. Water can be added to bind to sodium metal, where the combination of water and sodium metal can be removed.

4. Molecular Distillation

The oil is then moved to a distiller where it is heated to about 120° C. under vacuum. Mono esters and shorter carbon chain molecules move to the middle where they are cooled and drained, leaving longer carbon chains remaining as a concentrate. The process typically increases the key fatty acids by 100% during the first distillation; typically between 30-50% during the second distillation. The process can be repeated, although preferably the process is ideally only repeated once, as when oils undergo heat it can produce oxidation and degradation of the fatty acids in general. Oil waste is also increasing with repeated distillation, making the process less economical.

5. Reesterification (Reacylation)

The oil is then moved to a reesterification tank where the ethyl ester molecules are reconverted to the triglyceride form, which is the natural form of that fatty acid molecule. This natural triglyceride form comprises 98% of fats ingested by humans.

The esterification process takes place under low vacuum at about 80° C.

Glycerol is added to form the backbone of the glyceride molecules. Nitrogen can be added from the bottom of the tank to cause oil movement. Lipase enzymes are added as catalysts to facilitate the fatty acids binding to glycerol. The vacuum in the distillation tank removes the ethanol which was previously bound to the fatty acids. The enzymes used are lipases produced from bacteria or yeast. Perhaps the most effective enzymes are Candidan Antarctica lipase, and Chromobacterium Viscosum Lipase; other enzymes that can be used effectively are Psuedomonas, Mucor miehei, and Candida Cylindracea as well as other enzymes may also be used.

The reesterification process typically takes 24 hours, at which point the triglycerides typically reaches 60-65%, the remaining glycerides being diglycerides and monoglycerides. Around 3% of the fish oil will remain as ethyl esters, which can be removed together with the ethanol. Adding additional enzymes and/or continuing the enzymatic process can produce triglyceride molecule concentration of up to 99%. The 60-65% level is probably optimum from an economic point of view.

6. Winterization

The oil in triglyceride form is then moved to a cooling tank at 0° C., where saturated fats, in particular stearic acid are crystallized. The pulp is then pumped to a filter press, where the crystals are removed, essentially removing the vast majority of saturated fats from the oil. Depending on the amount of saturated fats in the oil, approximately 5-10% of the oil is lost during this process.

7. Bleaching

The oil is then removed to a bleaching tank at 60° C., where bleaching earth or bentonite earth is added to the oil. Any water in the oil evaporates due to the temperature. Any remaining impurities (trace minerals, etc) in the oil attach to the bentonite earth. The oil is then run through a bentonite earth filter to remove the bentonite earth together with the impurities.

8. Deodorization

Although not a necessary step, it is advantageous to move the oil to a deodorization tank. The tank contains low vacuum at 120° C. Steam is added at the bottom of the tank, which connects to color and odor molecules (oxidated matter, peroxides) which again travel into the vacuum system and into a residue container. This process gives the oil a neutral color with virtually zero taste and odor.

9. Mixing

The oil is then moved to a separate storage tank. Depending on the concentration of EPA and DHA desired, various batches can be mixed to yield the concentration desired for the final product.

10. Addition of Antioxidant

Antioxidants, in particular rosemary and mixed tocopherols can be added to the final oil to dramatically reduce the oxidation process.

11. Drumming

The oil is then drummed in stainless steel drums for storage and topped off with nitrogen to remove oxygen and minimize the potential for oxidation.

In some embodiments, the composition of the invention employs a mixture of omega-3 polyunsaturated fatty acids and/or derivatives that contain glycerides. For example, in one embodiment, the mixture contains about 35 wt. % triglycerides of EPA and about 25 wt. % triglycerides of DHA and about 10% other omega-3 fatty acids or derivatives thereof. In some embodiments, the mixture contains about 10 wt. % triglycerides of EPA, about 50 wt. % triglycerides of DHA and about 20% other omega-3 fatty acids or derivatives thereof, wherein the EPA and DHA are at least about 60% in the triglyceride form and the balance are at least about 90% of mono- and di-glycerides. In some embodiments, the mixture contains about 11% EPA and about 70% DHA, wherein at least about 60% of the combination of DHA and EPA are in the triglyceride form and the balance is at least about 90% mono- and di-glycerides. In another embodiment, the mixture can contain at least about 60 wt. % of a combination of EPA and DHA in a weight ratio of EPA:DHA of from about 1.4:1 to about 1.1:7 (for example, 1:2 to 1:5, 1:3, 1:4 or 1:7) wherein the combination is at least about 60% (e.g., at least about 80% or at least about 90% or at least about 98%) in the triglyceride form of the fatty acids and the balance is at least about 80% mono- and/or di-glycerides. In some embodiments, the combination is at least about 98% in the triglyceride form, with the balance being in the monoglyceride and/or diglyceride forms. Some of the above compositions are disclosed in copending U.S. patent application Ser. No. 12/015,488, filed Jan. 16, 2008 by Opheim. That patent application is incorporated by reference herein in its entirety.

Sources of the omega-3 polyunsaturated fatty acids or derivatives thereof include natural sources including, but not limited to, fish oil (e.g., cod liver oil or herring oil), flax seed oil, marine oils, sea oils, krill oil, algae and the like. Fish oil is a preferred source.

It is preferred to use a high quality source of omega-3 polyunsaturated fatty acids or derivatives thereof which is rich in omega-3 oils, preferably containing at least 70% omega-3 oils. The oil can also be rich in EPA and DHA moieties. Preferably, at least 75% of the omega oils contain EPA+DHA moieties, and more preferably 85% or more contain EPA+DHA moieties. The daily dose of omega-3 oils is about 1 to about 4 grams of omega-3 oil. One possible source is a balanced omega-3 formula such as Nordic Naturals, Inc.'s ProOmega nutritional supplement, which is 70% omega-3 oils of which 50.8% EPA moieties, 35.1% contains DHA moieties and 14.1% is other omega-3 polyunsaturated fatty acids or derivatives thereof.

One preferred source of omega-3 polyunsaturated fatty acids or derivatives thereof is Pro-DHA nutritional supplement sold by Nordic Naturals, Inc. It comprises 9% EPA derivative, 45% DHA derivative, and 4% other omega-3 polyunsaturated fatty acids or derivatives thereof. Still another preferred source of omega-3 polyunsaturated fatty acids or derivatives thereof is Nordic Naturals, Inc.'s Pro-DHA Elite which comprises 11% EPA derivative, 70.9% DHA derivative, and 4.9% other omega-3 polyunsaturated fatty acids or derivatives thereof.

In some embodiments, component (2) of the therapeutic compositions of the present invention contain polyunsaturated fatty acids or derivatives thereof other than omega-3 polyunsaturated fatty acids or derivatives thereof. For example, component (2) can contain omega-5, omega-6, omega-7, omega-9, and/or omega-11 polyunsaturated fatty acids and/or derivatives thereof.

The compositions of this invention can contain other ingredients besides those in components (1) and (2). These include, but are not limited to, flavor agents, fillers, surfactants (e.g., polysorbate 80 and sodium lauryl sulfate), color agents including, e.g., dyes and pigments, sweeteners, antioxidants and additional ingredients, such as vitamins, minerals and herbs.

### Flavor Agents

Useful flavor agents include natural and synthetic flavoring sources including, but not limited to, volatile oils, synthetic flavor oils, flavoring aromatics, oils, liquids, oleoresins and extracts derived from plants, leaves, flowers, fruits, stems and combinations thereof. Useful flavor agents include, e.g., citric oils, e.g., lemon, orange, grape, lime and grapefruit, fruit essences including, e.g., apple, pear, peach, banana, grape, berry, strawberry, raspberry, blueberry, blackberry, cherry, plum, pineapple, apricot, and other fruit flavors. Other useful flavor agents include, e.g., aldehydes and esters (e.g., benzaldehyde (cherry, almond)), citral, i.e., alpha-citral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanal (green fruit), 2-dodedenal (citrus, mandarin) and mixtures thereof, chocolate, cocoa, almond, cashew, macadamia nut, coconut, mint, chili pepper, pepper, cinnamon, vanilla, tooty fruity, mango and green tea. Mixtures of two or more flavor agents may also be employed. When a flavor agent is used, the amount employed will depend upon the particular flavor agent used. However, in general, the flavor agent can constitute from about 5% to about 50% by weight of the composition.

### Color Agents

Useful color agents include, e.g., food, drug and cosmetic (FD&C) colors including, e.g., dyes, lakes, and certain natural and derived colorants (such as caramelized sugars). Useful lakes include dyes absorbed on aluminum hydroxide and other suitable carriers. Mixtures of color agents may also be employed. When a color agent is employed, the amount used will depend upon the particular color agent used; however, in general, the color agent can constitute from about 0.5% to about 5% by weight of the composition.

### Sweetening Agent

Natural and/or artificial sweetening agents can also be added to the composition. Examples of sweeteners include sugars such as sucrose, glucose, invert sugar, fructose, and mixtures thereof, saccharin and its various salts (e.g., sodium and calcium salt of saccharin), cyclamic acid and its various salts, dipeptide sweeteners (e.g., aspartame), dihydrochalcone, and sugar alcohols including, e.g., sorbitol, sorbitol syrup, mannitol and xylitol, and combinations thereof. Natural sweeteners that can be employed include, but are not limited to, luo han, stevia or mixtures thereof. Luo han sweetener is derived from luo han guo fruit (siraitia grosvenorii) that is mainly found in China. It is about 300 times sweeter by weight than sucrose. Luo han is commercially available from, e.g., Barrington Nutritionals (Harrison, N.Y.). Stevia is derived from a South American herb, Stevia rebaudiana. It can be up to about 300 times sweeter than sucrose. Because luo han and stevia have such a sweet taste, only a small amount need be used in the composition. When a sweetening agent is employed the amount used will depend upon the particular sweetening agent used; however, in general, the sweetening agent can constitute from about 0.0005% to about 30%, by weight of the composition. When a sweetener having a very sweet taste, such as luo han or stevia, is used, small amounts such as about 0.0005% to about 0.1% (for example about 0.005% to about 0.015% or about 0.002% to about 0.003%) by weight can be used.

### Additional Ingredients

The compositions of the present invention can contain additional ingredients. Examples of such additional ingredients include, but are not limited to, vitamins, minerals and/or herbs.

As used herein, the term “vitamin” refers to trace organic substances that are required in the diet. For the purposes of the present invention, the term vitamin(s) include, without limitation, thiamin, riboflavin, nicotinic acid, pantothenic acid, pyridoxine, biotin, folic acid, vitamin B12, lipoic acid, ascorbic acid, vitamin A, vitamin D, vitamin E and vitamin K. Also included within the term vitamin are the coenzymes thereof. Coenzymes are specific chemical forms of vitamins. Coenzymes include thiamine pyrophosphates (TPP), flavin mononucleotide (FMM), flavin adenine dinucleotive (FAD), Nicotinamide adenine dinucleotide (AND), Nicotinamide adenine dinucleotide phosphate (NADP), Coenzyme A (CoA), Coenzyme Q10 (CoQ10), pyridoxal phosphate, biocytin, tetrahydrofolic acid, coenzyme B12, lipoyllysine, 11-cis-retinal, and 1,25-dihydroxycholecalciferol. The term vitamin(s) also includes choline, camitine, and alpha, beta, and gamma carotenes.

As used herein, the term “mineral” refers to inorganic substances, metals, and the like required in the human diet. Thus, the term “mineral” as used herein includes, without limitation, calcium, iron, zinc, selenium, copper, iodine, magnesium, phosphorus, chromium and the like, and mixtures thereof. Compounds containing these elements are also included in the term “mineral.”

As used herein, the term “herb” refers to organic substances defined as any of various often aromatic plants used especially in medicine or as seasoning. Thus, the term “herb” as used herein includes, but is not limited to, black currant, ginsing, ginko bilboa, cinnamon, and the like, and mixtures thereof.

In some embodiments, a dosage of the therapeutic compositions further includes antioxidants such as rosemary, vitamin E, astaxanthine, carnitine, and ascorbyl palmitate or other antioxidants known in the art for stabilizing fish oil and/or omega-3 polyunsaturated fatty acids or derivatives thereof.

The compositions of this invention are suitable for therapeutic and/or nutritional purposes in treating a subject in need of such treatment. As used herein, the term “subject” includes, but is not limited to, a non-human animal, such as a cow, monkey, horse, sheep, pig, chicken, turkey, quail, cat, dog, mouse, rat, rabbit, or guinea pig; and a human. Typically, the subject is a mammal, most typically a human.

The compositions of this invention can have beneficial effects on health, including, but not limited to, promoting, supporting or maintaining neurological, retinal or reproductive (e.g., fertility) health. Examples of neurological conditions that may be promoted, supported or maintained by the compositions of this invention include dementia, cognitive dysfunction, DHA or neurological deficiencies, overall brain health, mood, memory and concentration. The compositions may also promote, support or maintain brain and retina cell survival during both aging and the initiation and progression of neurodegenerative diseases. The compositions may also promote, support or maintain fertility and may strengthen composition of testis and sperm. In addition, the compositions may help those with digestive and/or fat absorption issues.

The amount of the composition of the invention that is effective will vary depending upon the condition being treated, and can be determined by standard clinical techniques. The precise dose to be employed will also depend on the relative amounts of the components of the compositions of the invention, route of administration, and the seriousness of the condition being treated and should be decided according to the judgment of the practitioner and each subject's circumstances.

The compositions of the present invention comprise components (1) and (2) wherein components (1) and (2) are present in amounts effective to promote, support or maintain neurological, retinal or reproductive health in a subject. The phrase “present in amounts effective to promote, support or maintain neurological, retinal or reproductive health in a subject” as used herein means that components (1) and (2) are used in an amount, individually and in combination, effective for a therapeutic, preventive or nutritional activity in a subject that promotes, supports or maintains neurological, retinal or reproductive health in the subject. By “promote, support or maintain neurological, retinal or reproductive in a subject” is meant the compositions help improve (or at least help maintain) the health of the subject's neurological, retinal or reproductive systems or functions. By “amount individually and in combination effective” is meant that each individual component is present in an amount sufficient to perform its function as well as the overall composition being in an amount sufficient to perform its overall function.

The form in which the composition of the invention is administered to the subject is not critical. Typically, the composition is administered as a liquid or in a capsule. Typically, the composition is administered in the form of individual doses. As used herein, the term “dose” includes both the case where the phospholipids omega-3 compound(s) are administered together (such as in the form of a capsule containing both components), and the case where the phospholipids and omega-3 compound(s) are administered separately (but, typically, at essentially the same time). In some embodiments, the composition of the invention is administered in the form of a daily dose. However, depending on the severity of the condition being treated, this may not be required, and the period between administration of the doses may be longer than one day. In addition, the term “administer” includes both the case where a third party administers the dose to the subject and the case where the subject self-administers the dose.

The present invention also includes a method of collecting phospholipids having 4,7,10,13,16,19-docosahexaenoic acid covalently bound thereto from herring roe, the method comprising:
(a) boiling a mixture comprising herring roe and water at about 100° C.,
(b) pressing the product of step (a) until excessive water is removed from the product and a presscake having a water content of 10% or less is formed,
(c) treating the presscake of step (c) with an alcohol until the phospholipids are released from it,
(d) collecting the released phospholipid molecules and distilling off the alcohol from the phospholipids to produce an oily product containing the phospholipid molecules, and
(e) deodorizing the product of step (d).

In some embodiments, if the viscosity of the product of step (d) is low enough, steam can be blown into the product of step (d) until impurities in the product rise to the surface. The impurities can then be removed under vacuum.

Since the fluidity of the product obtained in step (e) can be low, the viscosity of the product of step (e) can be reduced by adding fish oil to the product of step (e) to comprise from about 10% by weight to about 80% by weight of the total product. Adding fish oil will reduce the viscosity, allowing for the product to be encapsulated. Adding fish oil can also increase or decrease the overall concentration of DHA and/or EPA moieties in the product of step (e), depending on the concentration level of DHA and EPA moieties in the fish oil.

In some embodiments, the alcohol can be ethanol, methanol, propanol or isopropanol. In some embodiments, the alcohol is ethanol.

### Examples

A composition according to the present invention is prepared using the following ingredients in the amounts shown below. The phospholipids in the composition are derived from wild herring roe. A single 1500 mg serving of the composition (in the form of three 500 mg soft gel capsules) contains the ingredients shown in the table below in amounts also shown in the table. By way of comparison, a commercial composition derived from krill is also shown in the following table.

| | Wild Herring | | | |
|---|---|---|---|---|
| Ingredient | Roe Amount | | Krill Amount | |
| Total fatty acids**** | 930 | mg (62%) | 1020 | mg |
| EPA omega-3 glycerides (8%) | 120 | mg | 225 | mg* |
| DHA omega-3 glycerides (30%) | 450 | mg | 135 | mg* |
| Total omega-3 glycerides (40%) | 600 | mg | 450 | mg* |
| Total omega-5, 7, 11 (3%) | 45 | mg | (No claim) | |
| Total omega-6 (3%) | 45 | mg | 30 | mg* |
| Oleic acid (omega-9) (9%) | 85 | mg | 127.5 | mg* |
| Saturated (10-11%) | 160 | mg | 30 | mg* |
| Monounsaturated (11-12%) | 170 | mg | (No claim) | |
| Polyunsaturated (40%) | 600 | mg | 480 | mg* |
| Cholesterol (□3%) | 40 | mg | (Not listed) | |
| Astaxanthin (10 mcg/g) | 10 | mg | 2.25 | mg* |
| Vitamin A | Trace amounts | | 150 | IU* |
| Vitamin D | Trace amounts | | (None listed) | |
| | (near 13 IU) | | | |
| Vitamin E (natural mixed | Trace amounts | | 0.75 | IU* |
| tocopherols) | (near 4-6 IU) | | | |
| Total Phospholipids**** | 495 | mg | 600 | mg* |
| (30% of oil) | | | | |
| (25% of total DHA is bound to PL) | DHA PL** = | | DHA PL = | |
| | 112 | mg | 57 | mg |
| (29% of total EPA is bound to PL) | EPA PL*** = | | EPA PL = | |
| | 35 | mg | 96 | mg |
| Lyso-phosphatidyl choline (5% of | 25 | mg | 22.5 | mg (1.5%) |
| PLs) | | | | |
| Sphingomylin (1% of PLs) | 10 | mg | 3 | mg (0.2%) |
| Phosphatidyl choline (87% 0f PLs) | 400 | mg | 290 | mg (19.3%) |
| Phosphatidyl Inositol (□1% of PLs) | 7.5 | mg | 4.5 | mg (0.3%) |
| Phosphatidyl serine (□1% of PLs) | 5 | mg | 4.5 | mg (0.3%) |
| Phosphatidyl ethanolamine (5% of | 25 | mg | 3 | mg (0.2%) |
| PLs) | | | | |

*Based on product label claim
**DHA covalently bonded to a phospholipid
***EPA covalently bonded to a phospholipid
****All percentages and amounts are approximate and can vary slightly

Other ingredients include rosemary extract (lipid stabilizer antioxidant) and coloring agent (for the capsule shell).

Although the present invention has been described in considerable detail with reference to certain versions thereof, other versions are possible. Therefore the spirit and scope of the appended claims should not be limited to the versions presented herein.

What is claimed is:

1. A therapeutic composition comprising (1) phospholipids having 4,7,10,13,16,19-docosahexaenoic acid covalently bound thereto and (2) at least one omega-3 polyunsaturated fatty acid, or at least one pharmaceutically acceptable omega-3 polyunsaturated fatty acid derivative or mixtures thereof, wherein about 25% of the total 4,7,10,13,16,19-docosahexaenoic acid moieties in the composition are covalently bound to phospholipids, and wherein components (1) and (2) are present in amounts effective to support overall neurological health in a subject.

2. The therapeutic composition of claim 1 wherein component (1) further comprises phospholipids having 5,8,11,14,17-eicosapentaenoic acid covalently bound thereto.

3. The therapeutic composition of claim 1 or 2 wherein the phospholipids are derived from herring roe.

4. The therapeutic composition of claim 1 or 2 wherein component (2) comprises EPA, derivatives of EPA, DHA, derivatives of DHA or mixtures thereof.

5. The therapeutic composition of claim 4 wherein component (2) comprises a derivative of EPA.

6. The therapeutic composition of claim 4 wherein component (2) comprises a derivative of DHA.

7. The therapeutic composition of claim 4 wherein component (1) comprises a mixture of a derivative of EPA and a derivative of DHA.

8. The therapeutic composition of claim 4 wherein the derivatives of EPA and derivatives of DHA are selected from the group consisting of alkyl esters, glycerides and phospholipids and mixtures thereof.

9. The therapeutic composition of claim 8 wherein the derivatives of EPA and derivatives of DHA are glycerides.

10. The therapeutic composition of claim 1 or 2 wherein component (2) is a mixture comprising about 35 wt. % triglycerides of DHA and about 25 wt. % triglycerides of EPA.

11. The therapeutic composition of claim 1 or 2 wherein component (2) is a mixture comprising at least about 60 wt. % of a combination of EPA and DHA in a weight ratio of EPA:DHA of from about 1.4:1 to about 1.1:7, wherein the combination is at least about 60% in the triglyceride form of the EPA and DHA and the balance is at least about 80% monoglycerides, diglycerides or both.

12. The therapeutic composition of claim 11 wherein the combination is at least about 80% in the triglyceride form.

13. The therapeutic composition of claim 11 wherein the combination is at least about 90% in the triglyceride form.

14. The therapeutic composition of claim 11 wherein the combination is at least about 98% in the triglyceride form.

15. The therapeutic composition of claim 14 wherein the combination is at least about 98% in the triglyceride form and the remainder is monoglycerides, diglycerides or both.

16. The therapeutic composition of claim 11 wherein the combination comprises about 65 wt. % triglycerides of EPA and about 15 wt. % triglycerides of DHA.

17. The therapeutic composition of claim 16 wherein the combination is at least about 80% in the triglyceride form.

18. The therapeutic composition of claim 16 wherein the combination is at least about 90% in the triglyceride form.

19. The therapeutic composition of claim 16 wherein the combination is at least about 98% in the triglyceride form.

20. The therapeutic composition of claim 19 wherein the combination is at least about 98% in the triglyceride form and the remainder is monoglycerides, diglycerides or both.

21. The therapeutic composition of claim 11 wherein the combination comprises about 10 wt. % triglycerides of EPA and about 50 wt. % triglycerides of DHA.

22. The therapeutic composition of claim 21 wherein the combination is at least about 80% in the triglyceride form.

23. The therapeutic composition of claim 21 wherein the combination is at least about 90% in the triglyceride form.

24. The therapeutic composition of claim 21 wherein the combination is at least about 98% in the triglyceride form.

25. The therapeutic composition of claim 24 wherein the combination is at least about 98% in the triglyceride form and the remainder is monoglycerides, diglycerides or both.

26. The therapeutic composition of claim 1 or 2 further comprising a soft gelatin capsule into which components (1) and (2) are loaded.

27. The therapeutic composition of claim 26 wherein a daily dose of the therapeutic composition is delivered by an integral number of capsules.

28. The therapeutic composition of claim 1 or 2 wherein a daily dose of the therapeutic composition comprises 200 mg to about 6 grams of derivatives of DHA or derivatives of DHA plus derivatives of EPA wherein about 5% to about 99% of the derivatives are phospholipids and the remainder are derivatives other than phospholipids.

29. The therapeutic composition of claim 1 or 2 further comprising an antioxidant.

30. The therapeutic composition of claim 29 wherein the antioxidant is chosen from the group consisting of rosemary, vitamin E, astaxanthine, carnitine, and ascorbyl palmitate.

31. The therapeutic composition of claim 1 or 2 comprising the following ingredients in the following amounts:

| Ingredient | Amount | |
|---|---|---|
| Total Fatty Acids | 930 | mg |
| EPA omega-3 glycerides (8%) | 120 | mg |
| DHA omega-3 glycerides (30%) | 450 | mg |
| Total omega-3 glycerides (40%) | 600 | mg |
| Total omega-5, 7, 11 (3%) | 45 | mg |
| Total omega-6 (3%) | 45 | mg |
| Oleic acid (omega-9) (9%) | 85 | mg |
| Saturated (10-11%) | 160 | mg |
| Monounsaturated (11-12%) | 170 | mg |
| Polyunsaturated (40%) | 600 | mg |
| Cholesterol (□3%) | 40 | mg |
| Astaxanthin (10 mcg/g) | 10 | mg |
| Vitamin A | Trace amounts | |
| Vitamin D | Trace amounts | |
| Vitamin E (natural mixed tocopherols) | Trace amounts | |
| Total Phospholipids | 495 | mg |
| (25% of total DHA is bound to PL*) | DHA PL** = | |
| | 112 | mg |
| (29% of total EPA is bound to PL) | EPA PL*** = | |
| | 35 | mg |
| Lyso-phosphatidyl choline (5% of PLs) | 25 | mg |
| Sphingomylin (1% of PLs) | 10 | mg |
| Phosphatidyl choline (87% 0f PLs) | 400 | mg |
| Phosphatidyl Inositol (□1% of PLs) | 7.5 | mg |
| Phosphatidyl serine (□1% of PLs) | 5 | mg |
| Phosphatidyl ethanolamine (5% of PLs) | 25 | mg |

*Phospholipid
**DHA covalently bonded to a phospholipid
***EPA covalently bonded to a phospholipids.

32. A method of supporting overall neurological health in a subject comprising administering to the subject a dosage comprising the therapeutic composition of claim 1 or 2.

33. A method of supporting overall retinal health in a subject comprising administering to the subject a dosage comprising the therapeutic composition of claim 1 or 2.

34. A method of supporting overall reproductive health in a subject comprising administering to the subject a dosage comprising the therapeutic composition of claim 1 or 2.

35. A method of collecting phospholipids having 4,7,10,13,16,19-docosahexaenoic acid covalently bound thereto from herring roe, the method comprising:
(a) boiling a mixture comprising herring roe and water at about 100° C.,
(b) pressing the product of step (a) until excessive water is removed from the product and a presscake having a water content of 10% or less is formed,
(c) treating the presscake of step (c) with an alcohol until the phospholipids are released from it,
(d) collecting the released phospholipid molecules and distilling off the alcohol from the phospholipids to produce an oily product containing the phospholipid molecules, and
(e) deodorizing the product of step (d).

36. The method of claim 35 wherein the alcohol is ethanol, methanol, propanol or isopropanol.
